(19) United States
(12) Patent Application Publication
(10) Pub. No.: US 2003/0208252 A1
(43) Pub. Date: Nov. 6, 2003

(54) Mri ablation catheter

(76) Inventors: Gary S. O' Boyle, North Andover, MA (US); Charles A. Gibson III, Malden, MA (US)

Correspondence Address: Michael J Sweedler, Darby & Darby, Post Office Box 5257, New York, NY, 10150-5257, US

(21) Appl. No.: 10/275,727

(22) PCT Filed: May 14, 2001
(86) PCT No.: PCT/US01/15475

(51) Int. Cl. A61N 1/05

(52) U.S. Cl. 607/122; 606/41

(57) ABSTRACT

An ablation catheter which is compatible with MRI systems, including a shaft and a distal tip assembly, each being made of MRI compatible material, at least one electrode supported on the tip assembly, the electrode being made of MRI compatible material, and at least one MRI compatible wire connected to the electrode and extending from the electrode to the proximal end of the shaft, the wire being made of MRI compatible material.

### RELATED APPLICATION DATA

This application claims priority under 35 U.S.C. 119 from U.S. provisional application serial No. 60/204,419, filed May 12, 2000 which is hereby incorporated by reference in its entirety.

### FIELD OF THE INVENTION

This invention relates to ablation catheters which can be guided by magnetic resonance imaging.

### BACKGROUND OF THE INVENTION

Since its initial description in 1982, catheter ablation has evolved from a highly experimental technique to its present role as first-line therapy for most supraventricular arrhythmias including atrioventricular nodal reentrant tachycardia, the Wolff-Parkinson-White syndrome, and focal atrial tachycardia. More recently, the clinical indications for radio-frequency catheter ablation have expanded to include more complex arrhythmias that require accurate placement of multiple linearly-arranged lesions rather than ablation of a single focus. In contrast to catheter ablation of accessory pathways and atrioventricular nodal reentrant tachycardia, for which detailed mapping is required to identify appropriate sites for energy delivery, sites for catheter ablation of atrial flutter and atrial fibrillation, for example, are identified almost entirely on an anatomic basis. Therefore, the development of an alternative approach to guide placement of catheter ablation lesions based strictly on anatomical considerations and to confirm the location and presence of a continuous linear lesion is warranted.

Magnetic resonance imaging (MRI) may be an alternative to x-ray fluoroscopic techniques, as it offers several specific practical advantages over other imaging modalities for guiding and monitoring therapeutic interventions including; 1) real time catheter placement with detailed endocardial anatomic information, 2) rapid high-resolution three-dimensional visualization of cardiac chambers, 3) high resolution functional atrial imaging to evaluate atrial function and flow dynamics during therapy, 4) the potential for real-time spatial and temporal lesion monitoring during therapy, and 5) elimination of patient and physician radiation exposure. No studies to date, however, have evaluated the potential use of MRI to guide ablation therapy in the heart.

### SUMMARY OF EMBODIMENTS OF THE PRESENT INVENTION

It is an object of the invention to provide an improved method and apparatus or guiding an ablation and/or mapping catheter in connection with the treatment of supraventricular tachycardia, ventricular tachycardia, atrial flutter, atrial fibrillation and other arrythmias.

It is also an object of the invention to provide an ablation catheter which can be used with an MRI tracking and guiding system.

In one embodiment of the present invention, an ablation catheter for use with MRI is provided which consists of nonferrous or nonmagnetic materials for the components of the catheter which came in contact with the MRI tracking and guiding system, on the components which are internal to the patient. Note in b) there is no evidence of artifact and the catheter tip is clearly visualized in the right ventricle. Beginning with the first frame (a), the catheter is advanced through a jugular sheath into the superior vena cava. The catheter is then advanced into the right atrium (b-c), rotated 180 degrees (d) and advanced inferiorly into the Inferior Vena Cava (IVC) (e). In the final frame (f) the catheter was retracted to the lateral wall of the RA, which was the target site for catheter placement. Note the electrode-tissue interface is clearly visualized (frame f). The catheter may otherwise be of conventional design and either fixed curve or steerable. The catheter can be used with computer tomography (CT) which also requires the use of a nonmagnetic construction. Although preferred materials are indicated below, other nonmagnetic materials can be used.

### BRIEF DESCRIPTION OF THE DRAWINGS

FIGS. 1a and 1b are photographs acquired during RF delivery without (1a) and with (1b) radio frequency filters;

FIGS. 2a to 2f are photographs of catheter placement on the inferior-lateral wall of the right atrium;

FIG. 3a is a photograph of a Pre-ablation fast spin echo image including the electrode-tissue interface in the Right Ventricular Apex (RVA);

FIG. 3b shows the corresponding high amplitude intracardiac electrogram acquired during imaging;

FIGS. 4a and 4b are photographs of Pre and post-ablation FSE images following right ventricular apex Radio Frequency Ablation (RFA);

FIG. 4c is the corresponding mean intensity versus time data for a right ventricular apex lesion with a temporal resolution of approximately 2.0 minutes;

FIGS. 5a and 5b are photographs of pre and post lesion created images a) tip n position, and b) ablated tissue, including T I-weighted gradient echo images before (5a) and after (5b) peripheral 7 ml gadolinium-DTPA contrast agent;

FIG. 5c is the corresponding lesion intensity data with the temporal response for a fight ventricular lesion and adjacent segment of normal myocardium with the temporal resolution was approximately 30 seconds;

FIGS. 6a and 6b are photographs of fast spin echo images of the right ventricular free wall pre and 10 minutes post-ablation (6b);

FIGS. 6c and 6d are the corresponding intracardiac electrode tracings for FIGS. 6a and 6b respectively;

FIG. 7a is a right ventricular apex lesion with the spatial location of the intensity profile line;

FIG. 7b is the resulting intensity versus location data for a single point in time during the temporal assessment of the lesion;

FIG. 7c is a three-dimensional surface plot with the temporal and spatial development of right ventricular lesions, created by plotting several intensity profiles in time;

FIGS. 8a and 8b are photographs of direct visual comparisons of a right ventricular apex lesion appearance at gross examination (8a) and b) by MRI 8b;

FIG. 9 is a graphical comparison of a MR and a post-mortem lesion area;

FIG. 10 is a plan view of a steerable ablation catheter fitted with a distal assembly according to the invention;

FIG. 11 is a detailed perspective view of a control handle that may be used to steer the catheter of FIG. 10;

FIG. 12 is a plan view of the distal end of the catheter of FIG. 10 having a predetermined radius of curvature;

FIG. 13 is a plan view of the distal end of the catheter of FIG. 10, as modified to have a generally linear configuration distal to a predetermined radius of curvature;

FIG. 14 is an exploded perspective view of the distal tip assembly of FIG. 10;

FIG. 15A is an elevational view, partially in section, of the catheter of FIG. 10;

FIG. 15B is an elevational view, partially in section, of a more proximal portion of the catheter of FIG. 15A, and connects thereto along match line A-A;

FIG. 16 is a cross sectional view substantially taken along line 16-16 of FIG. 15A;

FIG. 17 is a cross sectional view substantially taken along line 17-17 of FIG. 15A;

FIG. 17A is a cross sectional view substantially taken along line 17A-17A of FIG. 15A illustrating the catheter of FIG. 10 as modified to have a generally straight segment distal of a predetermined radius of curvature when steered; and

FIG. 18 is a cross sectional view substantially taken along line 18-18 of FIG. 15B.

### DETAILED DESCRIPTION OF PREFERRED EMBODIMENTS

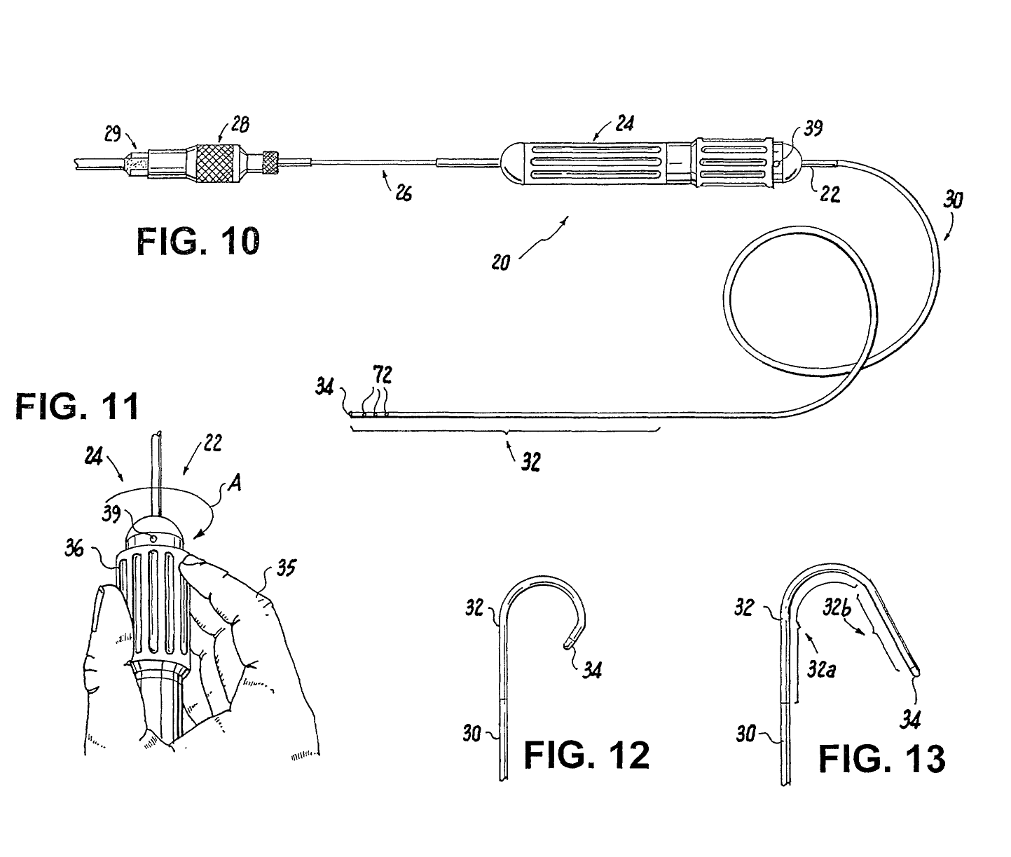

By way of overview and introduction, FIG. 10 illustrates a steerable ablation catheter 20 fitted with a snap-fit distal assembly 34. The catheter 20 includes a control handle 24 from which electrical wires 26 extend to a proximal connector 28. The catheter comprises a flexible, elongate shaft 30 which has a comparatively flexible distal segment or tipstock 32 connected to its distal end in conventional fashion. The shaft 30 and tipstock 32 are intended to be advanced through a patient's vasculature in conventional manner to the site to be treated. The catheter preferably has an overall length of approximately 115 cm for use in cardiac ablation procedures with the tipstock 32 extending from about four and a half to seven centimeters so that the catheter may be advanced through the femoral vein to a chamber within the heart, while the control handle 24 remains outside the patient to be manipulated by an operator 35 shown in FIG. 11. Different shaft 30 and tipstock 32 lengths can be chosen based on the procedure to be performed, the location at which the catheter is to be percutaneously introduced, and the anticipated path along which the shaft 30 must be steered. Preferably, the shaft 30 and tipstock 32 are made of a polyurethane tubing, the shaft 30 including a woven Dacron braid within the tubing to enhance stiffness and impart greater column and torsional strength to the shaft. The woven Dacron product has been available since the 1960s. For example, one type of woven Dacron is commercially available as catalog model number 200150 4F from C. R. Bard Inc., Glens Falls Operations.

The electrical wires 26 include conductive leads of copper or a copper alloy from a plurality of electrodes, temperature sensors, other electronic devices which may be included in catheter 20, or any combination of the above. The electrical wires 26 provide electrical signals to electronic components such as electrocardiogram (ECG) monitoring equipment and radio frequency (RF) RF energy sources directly through the connector 28, or through an intervening patient cable 29 (shown broken away).

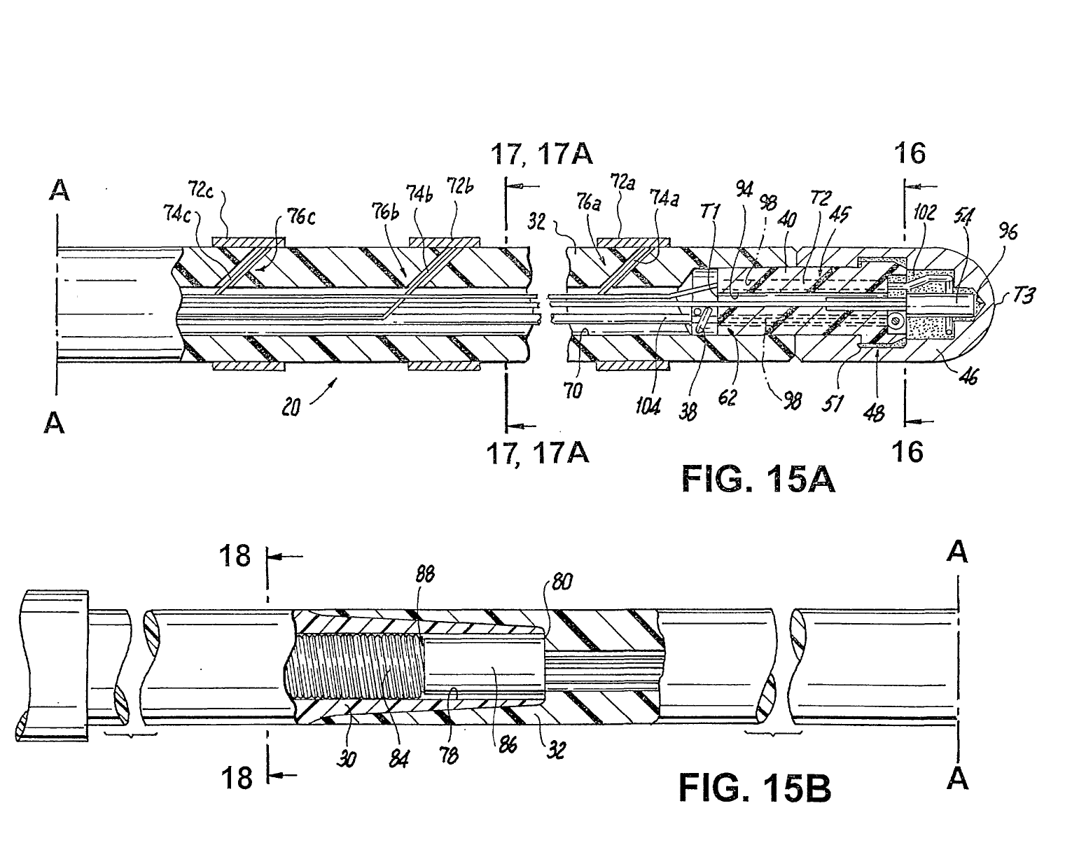

A knob 36 on the control handle 24 is rotatable relative to the handle (FIG. 11) by the operator 35 to cause a slideblock (not shown) within the control handle 24 to move away from a proximal end 22 of the shaft 30. A steering wire 38, which is slidably housed within the tipstock 32 and the shaft 30 (see FIG. 15A), is secured at its proximal end to the slideblock. The steering wire 38 is pulled proximally due to rotation of the knob 36, for example, in the direction of arrow A (FIG. 11). Conversely, the steering wire 38 advances distally when the slideblock moves toward the proximal end 22 of the shaft 30 as a result of rotation of the knob 36 in the opposite direction. The control handle 24 may be as described in U.S. patent application Ser. No. 08/518,521, filed Aug. 23, 1995 for Steerable Electrode Catheter to Bowden et al., the disclosure of which is hereby incorporated by reference as if set forth fully herein.

The steering wire 38 extends distally from the slideblock, through the shaft 30, to the distal tip assembly 34 where it is anchored, as described more fully below. the steering wire 38 is anchored to the distal tip assembly 34, a proximal pulling force on the steering wire 38 causes the tipstock 32 to deflect in a single plane and with a radius of curvature which is determined by the length and compressive strength of the tipstock 32, as shown in FIG. 13. The radius of curvature may be in the range of about two to four and a half centimeters. The steering wire 38 must have a tensile strength sufficient to overcome the compressive strength of the tipstock 32 to cause the tipstock 32 to deflect. When the knob 36 is rotated in a direction opposite to arrow A, the compressive forces on the tip stock are released to cause the catheter tip to return to its undeflected state. In the preferred embodiment, the steering wire 38 is a stainless steel wire having a pull strength of about 15.5 pounds.

The steering wire 38 is preferably guided eccentrically with respect to the longitudinal axis of the catheter 20, and more preferably guided eccentrically within the tipstock 32, so that the tipstock 32 will favor deflection in a known plane due to a wall thickness differential on either side of the steering wire 38 in the tipstock 32 (see FIGS. 15A and 17). The entire control handle 24 can be torqued by the operator 35 to steer the shaft 30 through the patient's vasculature. Additional steering wires can be provided, and a radius of curvature adjusting means can be provided in the manner described in the aforementioned U.S. patent application Ser. No. 08/518,521. The steering wires preferably are nylon (Spectra) cables.

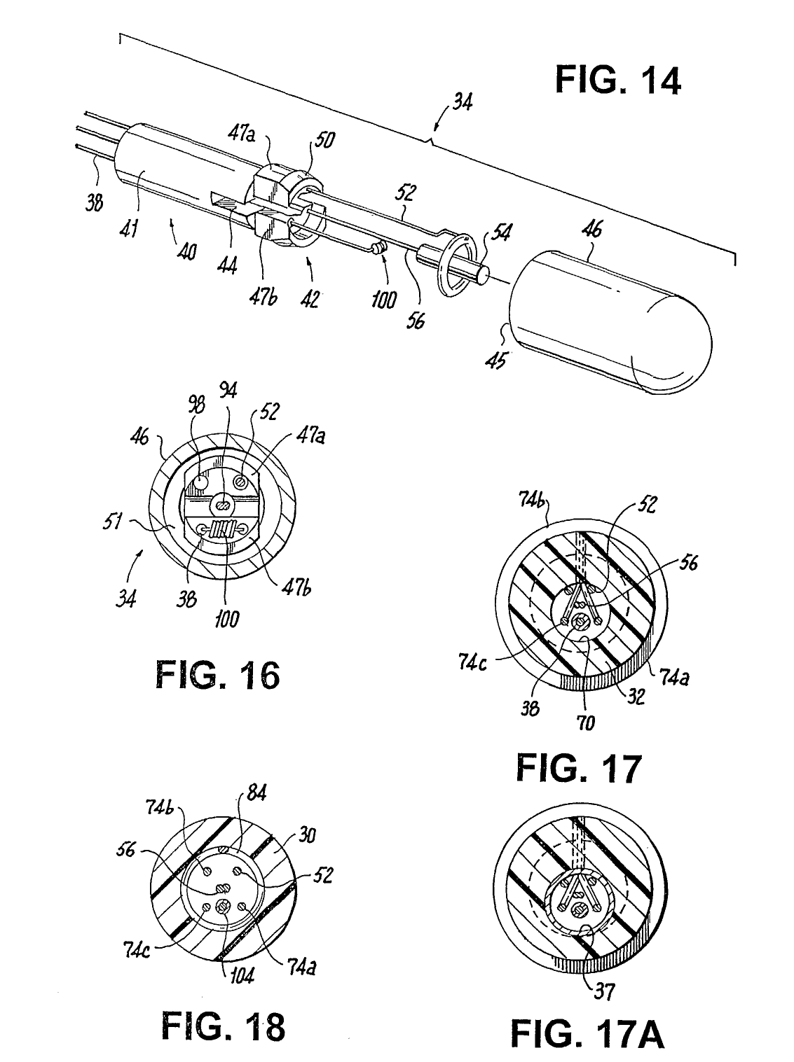

FIG. 14 is an embodiment of a steering catheter. Alternative structures of the steerability features of the catheters are within the scope of this invention. For example, the steerable catheter shown and described in U.S. Pat. No. 5,383,852 to Debbie Stevens-Wright et al., issued on Jan. 24, 1995, the entirety of which is hereby incorporated by reference can be implemented using the MRI compatible materials herein.

In FIG. 14, the shaft 30 has been modified to include a non-ferrous or magnetic hypotube 37 at its distal end which serves as a rigidifying element, for example, just proximal to the distal tip assembly 34 (FIG. 17a), so that rotation of the knob 36 causes deflection of the tipstock 32 with the distalmost portion 32b (FIG. 13) of the tipstock 32 remaining generally straight. A proximal portion 32a (FIG. 13) of the tipstock 32 which is clear of the hypotube 37 assumes a curve of a predetermined radius based on its length and its compressive strength. The tube or stiffening member 37 preferably extends about one to three centimeters along the catheter 20 and may be anchored to the steering wire 38, the distal assembly 34, or the distalmost used portion 32b of the tipstock. A stiffening wire or similar rigidifying element can be used in lieu of the hypotube 37.

The knob 36 preferably includes an indicator 39 (FIG. 11) which indicates that the knob has been rotated from its neutral position (where no force is applied to the steering wire 38). This means that a pulling force is being applied to the steering wire 38 and that the tipstock 32 is being deflected. The indicator 39 may be a tab affixed to the upper margin of the knob 36 which is visible through an aperture in the control handle 24 only when, for example, the slideblock is in a position proximate the proximal end 22 of the shaft 30. In this state, the tab is visible and indicates that no pulling force is being applied to the steering wire 38. Rotation of the knob 36 from the neutral position moves the indicator 39 out of registry with the aperture which indicates to the operator that a pulling force is being applied to the steering wire 38. The indicator 39 and knob 36 are preferably molded from a plastic material having a color which differs from that of the remainder of the control handle 24.

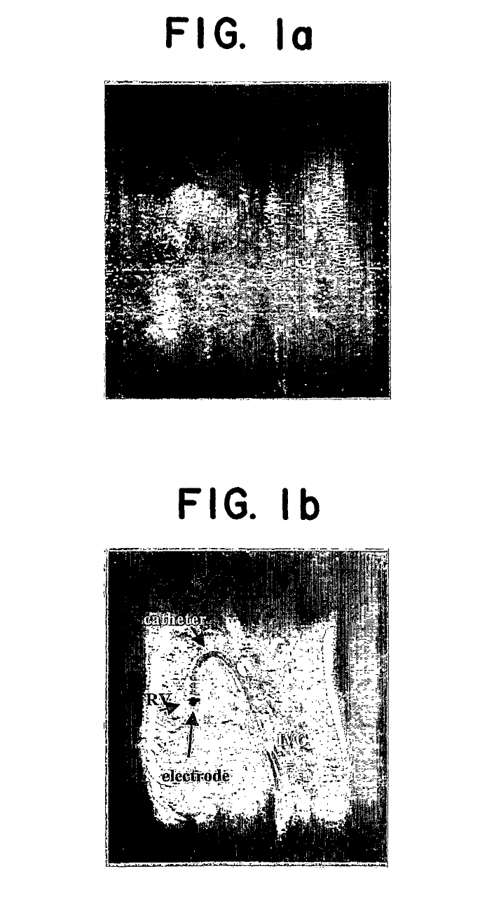

Turning now to FIG. 14, an exploded perspective view of the distal tip assembly 34 is shown. The distal assembly 34 comprises a core 40 which has a proximal portion 41 adapted to be received in the distal tip 34 of the tipstock 32, and a compressible head 42 at its distal end. The compressible head 42 includes anchor tabs 47a, 47b. The core 40 has a longitudinal slot 44 extending proximally from its distal face which permits the anchor tabs 47a, 47b to resiliently flex toward each other as the core 40 is received within an aperture 45 in a hollow non-magnetic (e.g. gold) ablation electrode 46 (FIG. 15a). Continued insertion of the core 40 into the ablation electrode 46 causes the anchor tabs 47a, 47b to snap into a groove 48 (FIG. 1 Sa) in the ablation electrode 46 which locks the core 40 and the ablation electrode 46 together. Due to tolerance control or other design considerations, the head 42 may remain in a partially compressed state even after the core and ablation electrode have snapped together so long as the two components interlock. The compressible head 42 includes a chamfered leading edge 50 which facilitates insertion of the core 40 into the aperture 45 of the ablation electrode 46 by camming the anchor tabs 47a, 47b together and thereby compressing the head 42 to a reduced profile. The groove 48 has a shoulder 51 (FIG. 15a) at its proximal edge which prevents the core 40 from being withdrawn from the ablation electrode 46 once the anchor tabs 47a, 47b have snapped into the groove 48 (FIG. 15A).

Alternatively, the core 40 and ablation electrode 46 may include a ratchet and pawl arrangement, or a generally annular projection made of an intrinsically compressible plastic such as polycarbonate or ULTEM®, shaped to mate with the groove 48 in the ablation electrode 46. For example, the annular projection may project about one to three nails on either side of the core 40, and the groove 48 in the ablation electrode 46 may be sized to receive the annular projection in an uncompressed state. In one embodiment, all that is important in these alternative configurations is that the core 40 and ablation electrode 46 interlock via a snap action. The core 40 is preferably made of a nonmagnetic material having a low temperature coefficient, such as the ULTEM® polyetheraide 1000 resin produced by the GE Plastics division of the General Electric Company, Pittsfield, Mass. The low temperature coefficient material provides thermal insulation between the ablation electrode 46 and the tipstock 32, and, preferably, the core 40 has a lower thermal mass than the ablation electrode. The provision of the core 40 between the tipstock 32 and the ablation electrode 46 reduces the likelihood of catheter damage during an ablation procedure which better ensures that a single catheter can be used for a given procedure, or perhaps reused (once sterilized) in subsequent procedures. The cap electrode 46 and the distal tip 34 of the tipstock 32 may be spaced from each other once the core 40 has been mounted in the distal tip 34 by a thin bead of epoxy, or by an annular ring on the core 40, disposed between its proximal end 41 and the compressible head 42. Further, a wider range of materials can be selected for the tipstock 32, including materials with melt-temperatures that are significantly less than the expected ablation temperature, such as polyurethane.

With further reference to FIGS. 14 and 15A, the distal assembly 34 preferably serves as an anchor for the steering wire 38 and also preferably houses a temperature sensor 54. The core 40 includes a central lumen 94 and several off-axis lumens 98 for conveying nonmagnetic wires 52, 56 from the ablation electrode 46 and temperature sensor 54, respectively, to the connector 28 (FIG. 10). The temperature sensor 54 is preferably a thermistor and may be positioned within a cavity 96 in the ablation electrode 46 about four to seven mils from the ablation electrode distal tip. A potting compound 102, for example, TRA-BOND FDA-2 epoxy made by Tra-Con, Inc. of Medford, Mass. may add rigidity to the entire distal assembly 34, as described below.

In FIG. 15A, there is seen a central bore 62 at the distal tip 34 of the tipstock 32. The central bore 62 is sized to fit the proximal end of the core 40. The tipstock 32 defines a lumen 70 for receiving the steering wire 38 and a surrounding teflon sheath 104 (FIGS. 15A-18), the temperature sensor conductive wires 56 (for example, made of copper/constantine), and the copper beryllium conductive wire 52 from the distal assembly 34. Mounted in spaced relation along the tipstock 32 are ring electrodes 72a, 72b, and 72c which may be sized for intracardiac ECG recording, mapping, stimulation, or ablation. Each ring electrode 72 may extend longitudinally about one half to four millimeters along the tipstock 32 from the ring electrode's proximal edge to its distal edge. The ring electrodes 72 are electrically connected to suitable components via copper/beryllium conductive wires 74a, 74b, and 74c which extend through respective apertures 76a-c in the side of the tipstock 32 into the lumen 70.

The ring electrodes 72 may be made of gold and spaced apart in the range of about one to five millimeters and may extend proximally sixty millimeters or more from the tip of the distal assembly 34 along the tipstock 32. For example, the ring electrode 74a may be two millimeters from distal tip 64 of the shaft 30, the ring electrode 74b may be spaced five millimeters from the proximal edge of the ring electrode 74a, and the ring electrode 74c may be spaced two millimeters from the proximal edge of the ring electrode 74b.

The tipstock 32 is connected to the distal end of the shaft 30 in conventional manner, preferably along complementary tapered and overlapping regions at their distal and proximal ends, respectively, by ultrasonic welding (FIG. 15B).

The lumen 70 of the tipstock 32 and the throughlumen 78 of the shaft 30 are in communication with each other. The lumen 70 is preferably disposed eccentrically relative to the longitudinal axis of the tipstock 32, so that proximally directed forces applied to the steering wire 38 cause the tipstock 32 to favor deflection in a predictable, single plane. Also, the eccentric lumen 70 creates an abutment 80 (FIG. 15b) in the vicinity of the union of the tipstock 32 and the shaft 30. In the preferred embodiment, a non-magnetic stiffening spring 84 (e.g. made of brass) extends from the proximal end 22 of the shaft 30 to the abutment 80. In the alternative, as shown in FIG. 15b, a nonmagnetic stiffening tube 86 may be interposed between the distal end 88 of the stiffening spring 84 and the abutment 80.

With reference now to FIGS. 14 and 16, the core 40 is interlocked to the ablation electrode 46, with the anchor tabs 47a, 47b of compressible head 42 snapped into the groove 48, just distal to the shoulder 51 (FIG. 1Sa). The anchor tabs 47a, 47b cannot be withdrawn beyond shoulder 51. Further, the steering cable 38 is shown looped through two of the off-axis lumens 98 in the core 40 and passing through a coil spring 100, which serves as a pressure reducing mechanism in the preferred embodiment to mitigate or eliminate a so called “cheese knife” effect in which the tensile force applied to the steering wire 38 causes the steering wire to cut into the distal face of the core 40. The coil spring 100 prevents the steering wire 38 from slicing the core by distributing a pulling force which may be applied to the steering wire 38 across the coils of the spring. Comparing FIGS. 15A and 16, the steering wire 38 is seen to extend distally through one of the lumens 98 in the core 40. In order to function 38 should pass through one of the 98 lumens to provide steering, i.e., in a loop joint, through the spring 100, and back through another of the lumens 98, preferably, to a point proximal of the core 40 where it is wrapped around itself to form an anchor for the steering wire 38. Preferably, the steering wire 38 is wrapped at least two times about itself. Favorable results have also been observed where the steering wire 38 is arranged to pass through one of the lumens 98, through the spring 100, and then partially back through another of the lumens 98, with the steering wire soldered to the spring 100.

FIG. 18 illustrates the eccentric lumen 70 in the tipstock 32 which causes a pulling force, which may be applied to the steering wire 38 via the control handle 24, to be directed eccentrically within the tipstock 32. The eccentric lumen 70 provides a reduced thickness lumen wall on one side of the steering wire 38. Further, the off-axis lumens 98 about which the steering wire 38 is anchored better ensures that the tipstock 32 repeatedly deflects in a predictable plane for reliable steering of the distal end of the shaft 30.

In FIG. 17A, the shaft 30 includes the hypotube 37 within the tipstock distal portion 32b. The hypotube 37 causes the distal end of the catheter to retain a generally straight configuration even when a pulling force is applied (see FIG. 4).

FIG. 17 is a cross section taken through the shaft 30 and illustrates the steering wire 38, conductive wires 56, conductive wire 52, and conductive wires 74 from the ring electrodes 72 extending proximally within the stiffening spring 84 toward the control handle 24.

The assembly of the distal tip assembly 24 is as follows. The plastic core 40 is preferably injection molded. The ablation electrode 46 is machined to have the desired overall dimension for the size of catheter with which it is to be used. The machining is preferably performed under computer control using a machine that can select a first drill bit to generally hollow out the ablation electrode 46, then a second, smaller bit to define the cavity 96, and finally to form the groove 48 using a key cutter, for example, by circular interpolation as understood by those of ordinary skill in the art of machining.

Conductive wire 52 is preferably wrapped like a lasso and resistance welded to the ablation electrode 46. Next, an epoxy which is thermally but not electrically conductive, for example, STYCAST® 2850 FT Epoxy Encapsulant, preferably mixed with Catalyst 24LV, both made by Emerson & Cuming Composite Materials, Inc. of Canton, Mass., is inserted into the central cavity 96 and the temperature sensor 54 bonded therein. The conductive wires 52 and 56 from the ablation electrode 46 and the temperature sensor 54 are threaded through the lumens 98, 94, respectively, either before or after their attachment to the ablation electrode 46.

The steering wire 38 is attached to the core by threading it in a U-shape through lumens in the core. In particular, the steering wire 38 is threaded through one of the off-axis lumens 98, through the coil spring 100, and then through another of the off-axis lumens 98. The steering wire may extend to a point proximal of the core 40 at which location it may be wrapped about itself to complete its anchoring, or it may terminate after the U-shaped bend within one of the lumens 98 and instead be soldered or brazed to the coil spring 100. Preferably, a teflon coated steering wire 38 is selected, the portions of the steering wire 38 that are anchored to the core 40 and the control handle 24 preferably being stripped clear of the teflon. Teflon is difficult to bond and is removed to anchor the exposed steering cable. Alternatively, a lubricous sleeve such as teflon may be bonded to the steering wire 38 to reduce the frictional forces that are imparted by the walls of lumens 70, 78 when the steering wire is moved and electrically insulate the steering wire. A second steering wire 38A may be threaded through lumens 98 disposed on the opposite side of the central lumen 94.

After the conductive wire 52, the temperature sensor 54, and the steering wire 38 have been suitably attached, the ablation electrode 46 may be filled with a potting compound 102 such as FDA-2 epoxy and the core and ablation electrode snapped together in the manner previously described. The snap action of the core 40 and ablation electrode 46 is both audible and tactile. Further, the steering wire, thermistor wires, and ablation electrode wire are received without any twisting action unlike other known methods of making an ablation catheter. Moreover, the potting compound 102 electrically and thermally isolates the steering wire 38 from the ablation electrode 46.

Next, the steering wire 38, conductive wires 56, and conductive wire 52 may be threaded through the lumen 70 and throughlumen 78 to the control handle 24 to assemble the distal tip assembly 34 on the catheter 20. The proximal end of the core 40 can be coated with an epoxy prior to insertion into the central bore 62 at the distal end of the tipstock 32. A thin bead of epoxy (not shown) may space the cap electrode 46 from the distal tip 64 of the tipstock 32 when the distal assembly 34 is mounted to the catheter 20, or the core 40 may include an annular ring which spaces the ablation electrode 46 from the distal tip 64 when the core is inserted into the distal tip. The assembly is completed by attaching the steering wire 38 to the slideblock and the conductive wire 52, 56, and 74 to respective ones of wires 26.

Using a catheter of the type described above, a series of experiments were conducted 1) to develop and characterize a novel MR ablation system capable of guidance, delivery, and monitoring of cardiac radio-frequency thermal therapy, 2) to quantify temporal and spatial MR signal changes in cardiac tissue following radio frequency induced thermal damage, and 3) to correlate MR lesion size with postmortem lesion size and quantitative histologic markers of cellular death.

Methods

Magnetic Resonance Imaging System

Experiments were performed in a short 1.5 T closed-bore real-time interactive cardiac MR1 system (Signa LX, General Electric Medical Systems, Milwaukee, Wis.) using a standard cardiac phased array coil. This new system overcomes the limitations of conventional MR systems that rely on static scanning protocols by providing rapid data acquisition, data transfer, image reconstruction and real-time interactive control and display of the imaging slice, while allowing for direct access to the groin or neck for catheter insertion and manipulation. The realtime hardware platform consists of a workstation and bus adapter that can be added to conventional scanners. Details of this system have been described elsewhere (See Yang P C, Kerr A B, Liu A C, Liang D H, Hardy C, Meyer C H, Macovski A, Pauly J M, Hu B S. New real-time interactive cardiac magnetic resonance imaging system complements echocardiography. J Am Coll Cardiol 1998;32(7):2049-56; and Kerr A B, Pauly J M, Hu B S, et al. Real-time interactive MRI on a conventional scanner. Magn Reson Med 1997;38:355-67.).

Radiofrequency Catheter Ablation System

Radiofrequency ablation was performed using a standard clinical RF generator (Atakr®, Medtronic, Minneapolis, Minn.) with open loop control. The generator was located outside the scan room and was electrically interfaced to the animal via the above described ablation catheters.

A technical limitation of radio frequency energy delivery and electrophysiologic signal acquisition in the scanner is electromagnetic interference. While the frequency of the radio frequency generation unit (−500 kHz) is well below the 64 MHZ proton precession frequency at 1.5 T, higher harmonics of the radio frequency signal can produce significant image degradation. To overcome this problem, special RF filters and shielding were designed and constructed to suppress these harmonic signals and permit simultaneous RF ablation and electrophysiological monitoring during imaging. These multi-stage, low-pass filters consist of an arrangement of non-magnetic electrical components that achieve a cut-off frequency of approximately 1 OMHz. The output from the RF generator is directed to the ablation catheter through these fully shielded filter assemblies that pass through an electric patch panel between the scan and console rooms. The dispersive ground electrode consists of a large conductive adhesive pad that is attached to the skin of the animal to complete the circuit. Intracardiac electrogram tracings were acquired using the same catheters via a similar 12-channel shielded filter box and were recorded using automated data acquisition software. The effect of the RF ablation signal on image quality is shown in FIG. 1. The left panel represents an image acquired during RF delivery without filtering while the image on the right shows the same slice during RF delivery with filtering. Note that there is no evidence of noise or artifact and the tip of the catheter is clearly visible in the right ventricular apex (arrow).

Animal Preparation and Experimental Protocol

All animal protocols were reviewed and approved by the Animal Care and Use Committee at the Johns Hopkins University School of Medicine and conformed to the guidelines published in the “Position of the American Heart Association on Research Animal Use.” Six mongrel dogs weighing 28-36 kg were pre-medicated with a 10 mg intramuscular injection of ketamine and maintained on 80% oxygen and 1% isofluorane gas throughout the experiment using a Narkomed Anesthesia ventilator (North American Draeger, Telford, Pa.). Surface electrocardiogram (ECG) leads 1,2 and 3 were monitored continuously throughout the experiment. Using standard techniques, 8 Fr introducer sheaths were placed in the right jugular vein for catheter access and in the right femoral vein for administration of fluids and medication.

Under MR guidance, a 7F non-magnetic single electrode ablation catheter was positioned at the inferior lateral wall of the right atrium in three animals to determine the accuracy of catheter localization under MR guidance (no ablation). In the same animals, two ablation sites in the right ventricle (apex and free wall) were targeted for ablation from a right jugular vein access using a fast gradient recall echo (FGRE) sequence (TR=5 ms, TE=1.2 ms, field of view=22 cm, slice thickness=7 mm, 256×128 matrix, tip angle=13 degrees, readout bandwidth=31.0 kHz). Once electrode-wall contact was visualized and confirmed by intracardiac electrogram tracings, the catheter was imaged to isolate the optimal tomographic slice containing the catheter electrode. After baseline images were acquired for this slice prescription, RF ablation was performed in the right ventricle between the distal electrodes and a large surface area skin patch at a power of 20 W for 60 seconds. To avoid electrode coagulum formation, impedance was monitored by an automatic open-loop feedback system that terminates RF delivery if the impedance exceeds 220 ohms. The isolated slice and two immediately adjacent slices were then subsequently imaged once every two minutes over 20 minutes with a T2-weighted fast spin echo (FSE) sequence (TR=2XRR, TE=68 ms, ETL=16, field of view=22 cm, slice thickness=7 mm, 256×192 matrix, readout bandwidth=62.5 kHz) to monitor temporal signal change and lesion growth over time. Following this imaging series (30 minutes post ablation), 0.3 ml/kg of gadolinium-DTPA was administered as a bolus injection into an intravenous line and the same slice was imaged every 30 seconds over 12 minutes using the same T I-weighted gradient echo sequence described above with a tip angle of 40 degrees.

Postmortem Exam

Following experiments, the animal was sacrificed by anesthesia overdose and the heart was excised and sectioned through the right ventricular lesion into slices corresponding to the tomographic MR imaging slices. Lesion location, morphology, width, length and transmural extent were determined and recorded at gross examination and right ventricular lesions were photographed and matched with the corresponding T2 and contrast enhanced T1-weighted lesion images. Sections from thermally damaged tissues were bisected longitudinally and submitted for histologic staining (Masson's trichrome and hematoxylin-eosin). Specimens were then analyzed under light microscopy at 40× to characterize global morphologic changes (9) (e.g., delineated cellular junctions and nuclei, and interstitial edema) for determination of the degree of heat induced cellular damage and necrosis.

Data Analysis

To determine the temporal response of cardiac tissue following RF delivery, lesion signal intensity, length, width and area were measured directly from MR images using an off-line quantitative analysis package (Image Tool, Scion Image, Bethesda, Md.). Each parameter was measured 10 times for each time frame from baseline to 20 minutes post-ablation. Mean signal intensity from region of interest (ROI) measurements was then normalized (mean ROI signal intensity at time t divided by the baseline signal intensity) and plotted as a function of time. A similar method was used following gadolinium injection on T1-weighted imaging. Additionally, IEGMs were analyzed pre and post-ablation for changes in signal amplitude and waveform shape. For accurate and consistent determination of MR lesion size by free hand planimetry, it was necessary to establish quantitative exclusion criteria regarding the spatial distribution of signal intensity through the lesion. This was achieved by rejecting pixel values around the periphery of the lesion that were less than the normal myocardium signal intensity plus one standard deviation of the background noise as determined from ROI intensity measurements. Lesion parameters at gross examination were measured independently of MR hand-planimetered lesion parameters and compared.

Statistical Analysis

Changes in mean signal intensity, intracardiac electrocardiogram amplitude and tissue birefringence intensity pre- and post-ablation were considered significant at a level of p<0.05 using a paired t test. Lesion area measurement comparisons between MR and gross examination were analyzed by linear regression using a paired t test at a level of p<0.05.

Results

Catheter Placement

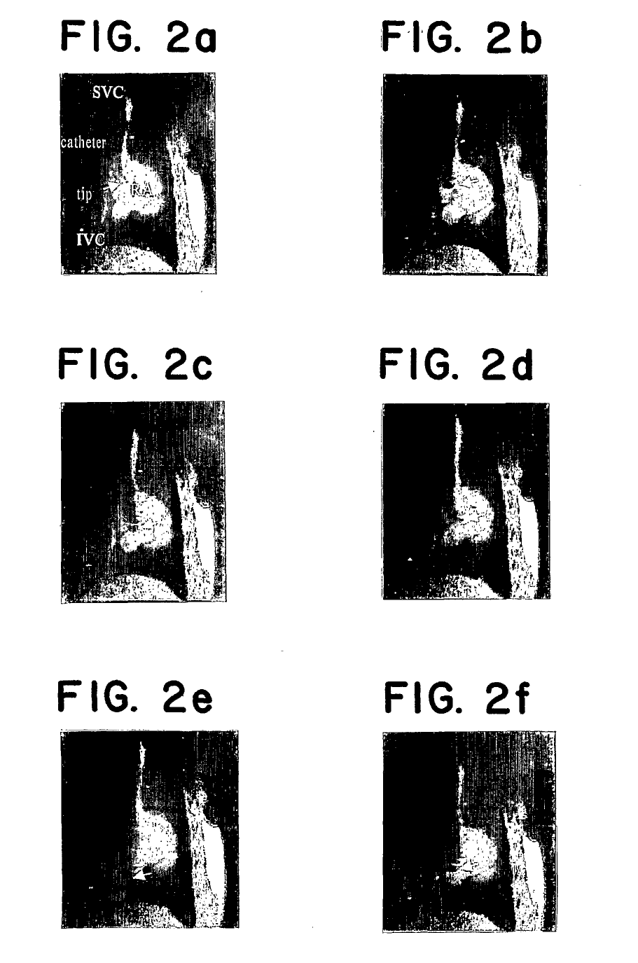

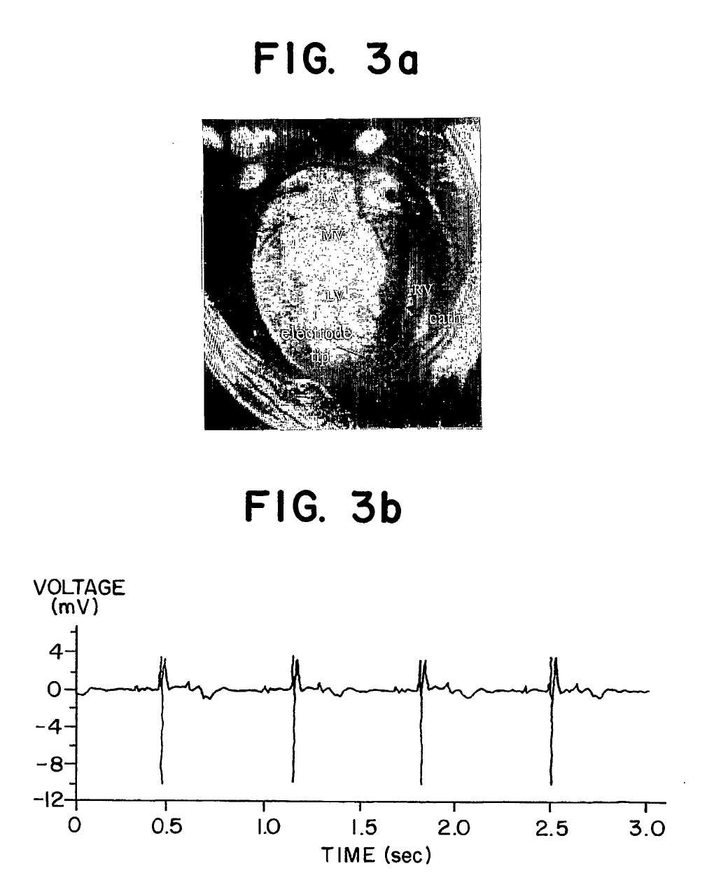

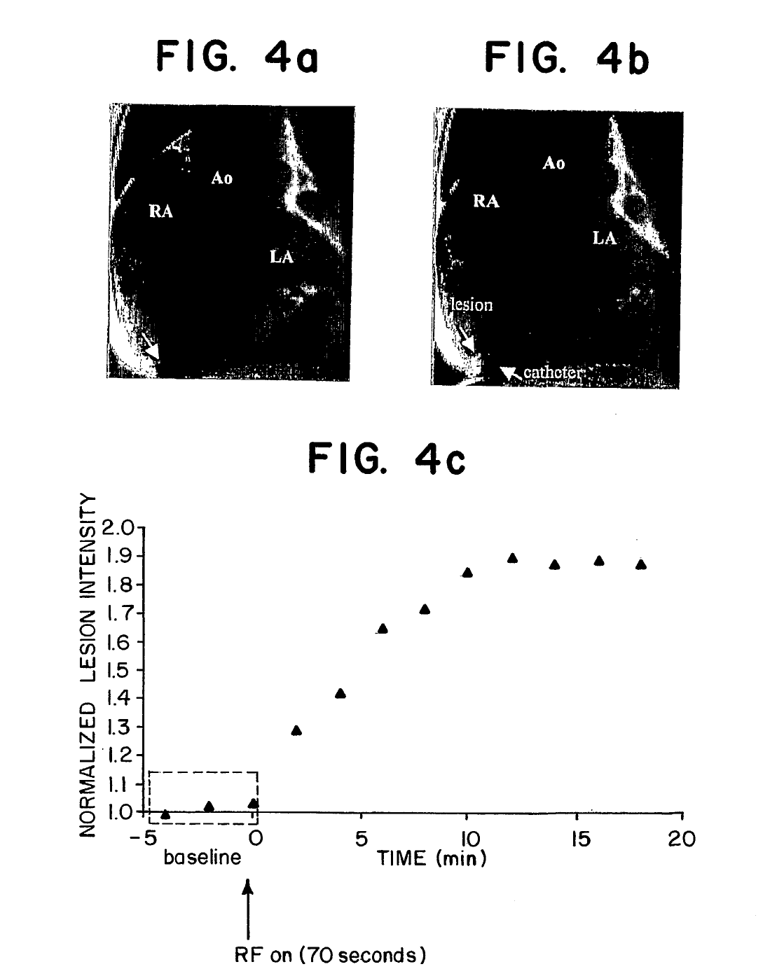

A MR fluoroscopy sequence was used to successfully position the non-steerable catheter at atrial and ventricular target sites in all animals. In three animals, MR catheter placement was attempted to target the inferior lateral wall of the right atrium from a jugular access (FIG. 2). Images were acquired without breath-hold once every heart beat with one-second updates. Details of the right atrial anatomy could be appreciated in all animals as several major endocardial anatomic landmarks were successfully identified, including the superior and inferior vena cava, atrial septum, right atrial appendage, coronary sinus, eustachian ridge, fossa ovalis and tricuspid valve. The catheter remained in the imaging plane throughout the entire navigation sequence in 2 of 3 animals. Contact between the electrode and tissue could be visualized without significant electrode artifact (FIG. 2f) and inferior-lateral wall catheter localization was successful and reproducible in each animal. Right ventricular ablation sites were successfully targeted in all animals and the electrode-tissue interface was clearly visualized during FGRE imaging (FIG. 3a) with visual catheter stability confirmed by high fidelity IEGMs (amplitude=10.7 mV) as shown in FIG. 3b.

MRI Lesion Visualization and Temporal Signal Response

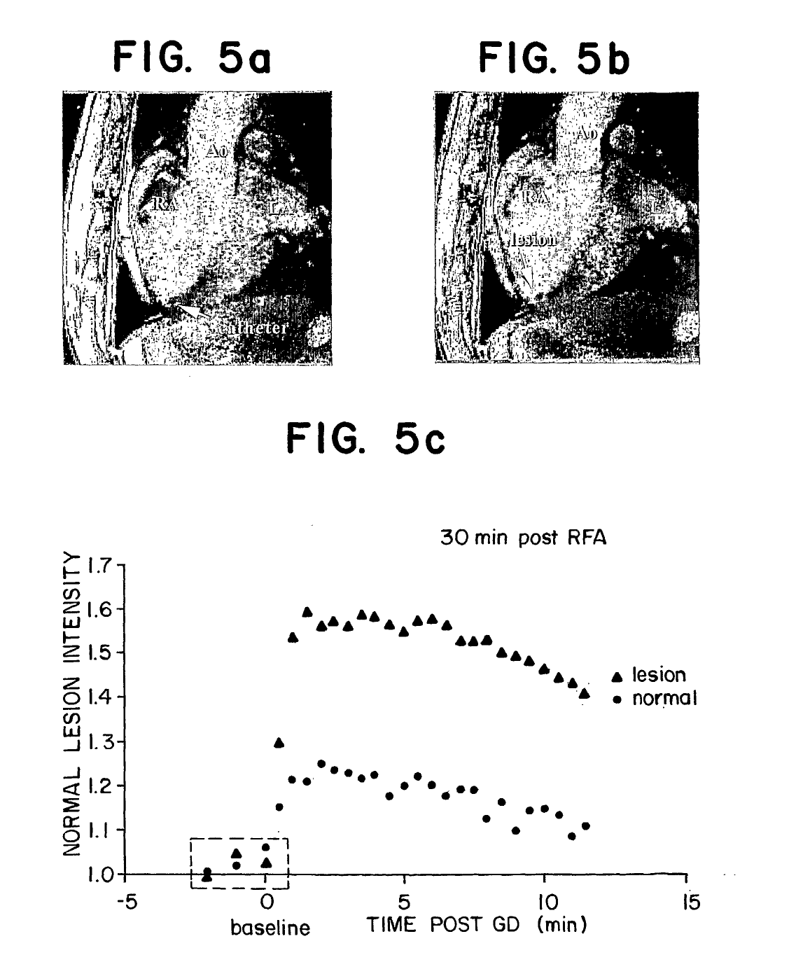

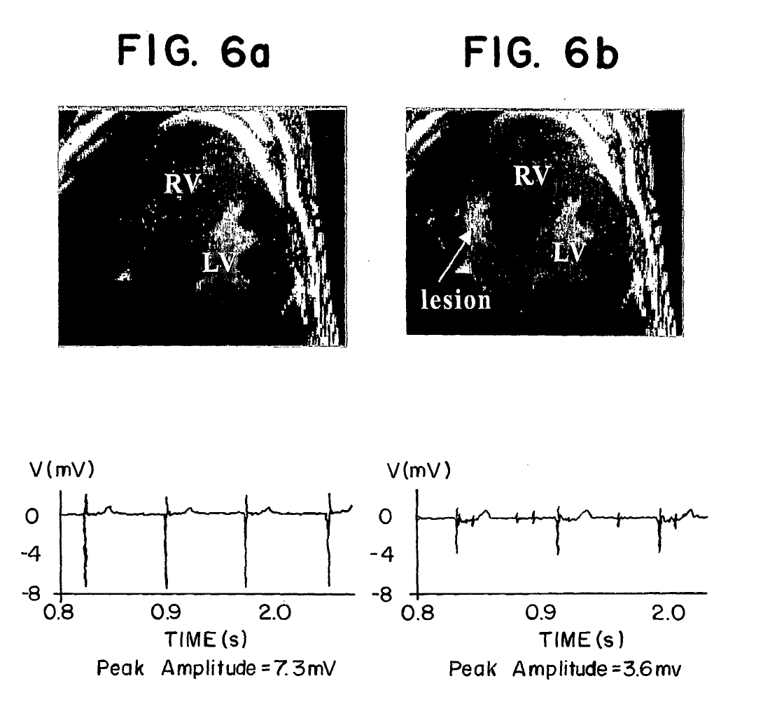

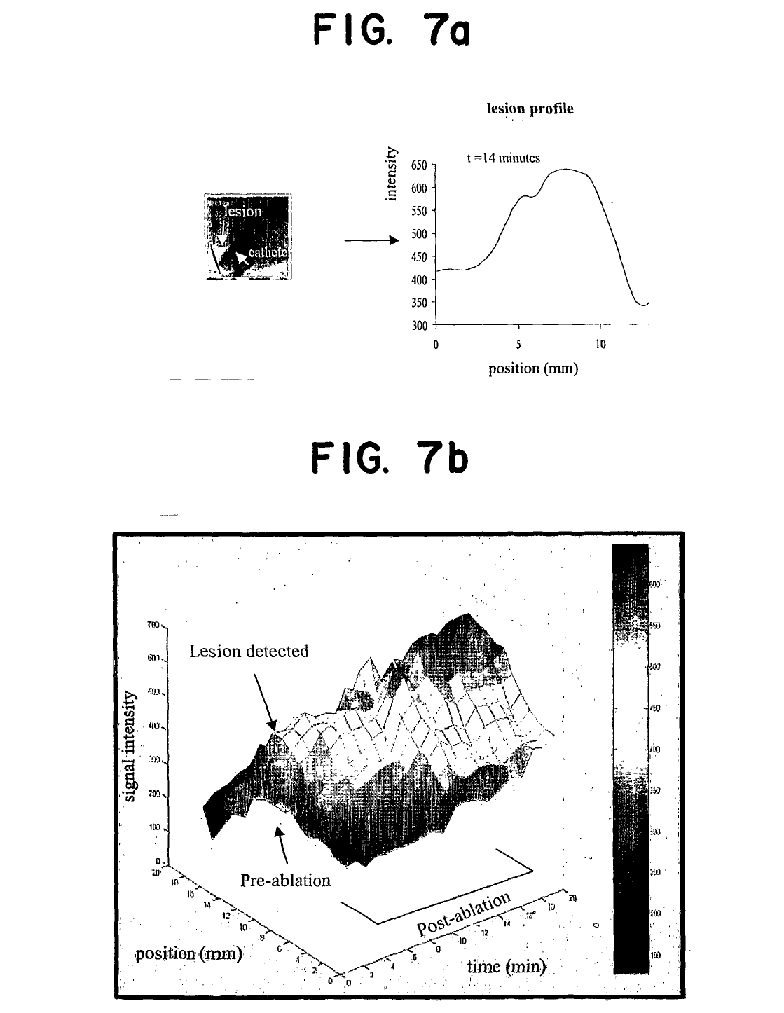

Lesions were successfully created and visualized at right ventricular target sites in all animals. Ventricular lesions appeared as clearly delineated hyperintense regions directly adjacent to the ablation catheter tip and were detectable 2 minutes following the RF delivery (FIG. 4). The lesion signal intensity response is shown in FIG. 4c at a temporal resolution of approximately 2 minutes, with the first three time points representing baseline myocardial signal intensity pre-ablation. Mean intensity increased linearly over the first 10 minutes and was then followed by a plateau. Mean FSE signal intensity 15 minutes post ablation was 1.9±0.4 times greater than the baseline myocardial intensity (p<0.05) and the mean time to signal plateau was 12.2±2.1 minutes. FSE imaging time averaged 1.7±0.3 minutes per slice. Approximately 30 minutes following this sequence of images, T I-FGRE images of the same tomographic slice were acquired before and following 7 ml peripheral gadolinium injection (FIGS. 5a,b). The lesion border was clearly demarcated 60 seconds following contrast injection. Intensity versus time data for the contrast-enhanced lesion (temporal resolution=30 seconds) indicated a rapid initial uptake of gadolinium and a gradual washout over the next several minutes (FIG. 5c). Data for an adjacent region of undamaged myocardium indicated a significantly lower level of enhancement that followed a similar temporal course over the imaging interval (L 13±0.12 versus 1.55±0.16, p<0.05). Under MR fluoroscopy guidance, the catheter was moved from the right ventricular apex and repositioned on the right ventricular free wall. FSE images before and after RF delivery are shown in FIG. 6 with the respective IEGM tracings. A large lesion was visualized directly adjacent to the ablation catheter tip and demonstrated a temporal response similar to those measured in right ventricular apex lesions, with peak intensity occurring 11.2 minutes post-ablation. Considering data from all animals, IEGM amplitude decreased from a mean pre-ablation value of 10.3±3.1 mV to 2.2±3.3 mV following RF delivery (p<0.05). FIG. 7 is a series of lesion profile plots that characterize the spatial and temporal formation of ventricular lesions. A lesion profile is simply a plot of signal intensity over a fixed spatial domain passing though the lesion, as illustrated by FIG. 7a for a single time frame. The three-dimensional surface plot represents a series of these profiles in time, where the z-axis represents the color-coded signal intensity and the x and y-axes represent position and time following RF delivery, respectively. The lesion grew dramatically in signal intensity and size from the baseline level shown by the arrow. Maximum signal intensity and lesion area were achieved 12.2±2.1 and 5.3±1.4 minutes following RF delivery, respectively.

Correlation With Gross and Histopathologic Examination

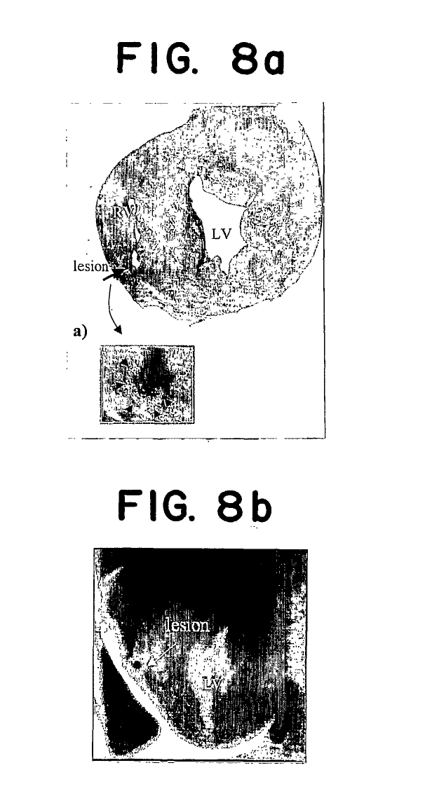

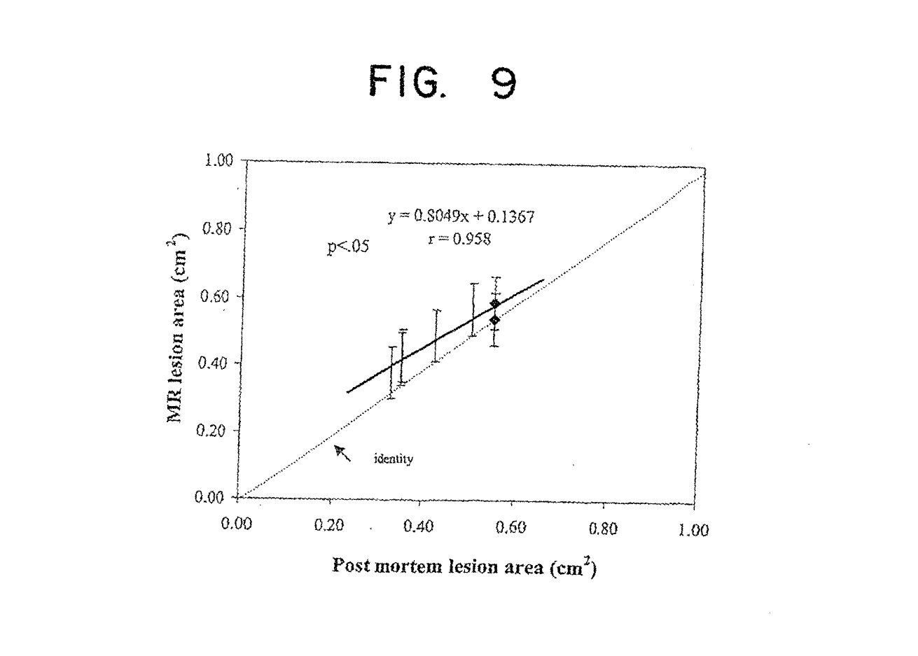

Direct visual comparison of right ventricular apex lesions at gross examination and those derived by MR 10 minutes post-ablation demonstrated similar lesion geometries (FIG. 8). Lesion width and length measured at gross exam correlated well with MR-derived measurements (width: 6.7±0.5 versus 7.1±0.9 nim, p<0.05, length: (9.4±1.5. versus 9.9±0.9, p<0.05). MR lesion depth could be assessed quantitatively in three animals and also agreed well with gross exam measurements (depth: 3.4±2.1 versus 3.1±1.2 mm. p<0.05). All lesions were comprised of a series of three concentric elliptical zones of damage: a dark inner portion representing a region of coagulative necrosis (zone 1); a surrounding pale peripheral circular zone of hemorrhage and inflammatory cells that extended approximately 4 mm from the center of the lesion (zone 2); and an outermost area consisting of a thin purple rim extending an additional 2-3 mm (zone 3). Low power trichrome-stained histologic specimens clearly demarcated the pathologic lesion from native undamaged tissue in all animals. A strong agreement and correlation was observed (FIG. 9) between the spatial extent of right ventricular MR derived lesions and the actual extent of damage measured at gross and histopathologic examination (55.4±7.2 versus 49.7±5.9 mm, r=0.958, p<0.05).

Main Findings

This study concerns a novel MRI-compatible interventional electrophysiology hardware system in conjunction with a newly developed real-time interactive cardiac MRI system to characterize the temporal and spatial development of cardiac lesions following radiofrequency ablation. This finding indicate that: 1) MR images and IEGMs can be acquired during radiofrequency ablation therapy using specialized radiofrequency filters; 2) nonmagnetic MR compatible catheters can be successfully placed at right atrial and right ventricular targets using fast MR imaging sequences with interactive scan plane modification; 3) regional changes in ablated cardiac tissue are detectable and can be visualized using FSE and FGRE images; 4) the spatial extent of heat induced necrosis can be accurately quantified by MRI immediately following thermal damage; and 5) lesion transmurality can be assessed. These results may have significant implications for the guidance, delivery, and monitoring of cardiac ablation therapy by interventional MRI.

MR Guided Catheter Placement

Right atrial and ventricular sites were successfully targeted in all animals with nonsteerable catheters using real time MR fluoroscopy pulse sequences. The high-resolution images of endocardial anatomy combined with the ability to interactively modify the scan plane considerably improved targeting and accurate lesion placement since standard fluoroscopic views could be defined in real-time using a graphical interface. Accurate atrial catheter placement has clinical importance for the study of a variety of supraventricular arrhythmias as the relationship between endocardial anatomy and arrhythmia substrate becomes increasingly appreciated. Current techniques to map and identify arrhythmogenic foci are based upon low-resolution voltage maps generated by catheter movements under x-ray fluoroscopy. In addition to limited anatomic information, catheter manipulation under x-ray fluoroscopy can be arduous and poorly reproducible. Anatomic MRI guided electrophysiologic mapping may significantly improve the localization accuracy of critical arrhythmogenic substrate.

Another very important feature of MR guided catheter placement is the ability to visualize the electrode-endocardial tissue interface, which has been shown to increase lesion size by improving the efficiency of RF tissue delivery. While traditional indicators of electrode contact such as fluoroscopic catheter stability and intracardiac electrogram amplitude are useful, these parameters are relatively insensitive indicators of electrode-tissue contact. An important limitation of passive MR catheter tracking, however, is the need to manipulate the catheter within the imaging slice (typically 5-10 mm wide), which may be especially difficult during catheter placement in geometrically complex vessels and cardiac chambers where catheter curvature and loops are common. This places demands on the MRI system to permit rapid sweeping through slice locations. To improve the accuracy of MRI catheter positioning, we are currently developing active tracking techniques that provide the x,y,z space coordinates of the ablation catheter tip superimposed upon interactive three-dimensional images of the atrial chambers.

In Vivo Lesion Visualization

Perhaps one of the greatest advantages of MRI guided therapy is the ability to visualize and monitor lesion formation with high temporal and spatial resolution. In this study, right ventricular lesions were created and visualized using both a T2-weighted fast spin echo sequence and a gadolinium-enhanced T1-weighted fast gradient recall echo sequence. Lesions imaged using FSE appeared acutely as elliptical, hyperintense regions directly adjacent to the catheter tip, however, zones of reversible and irreversible damage were not visible. FGRE contrast-enhanced lesions 30 minutes ablation showed rapid uptake of gadoliniurn following injection and represented the affected area similar to FSE images. The mechanisms of lesion enhancement for these two sequences are quite different and may lend insight into the biophysics of in vivo tissue damage and lesion formation.

Fast Spin Echo imaging. MR1 is able to detect one or more specific changes in T1 and T2 relaxation parameters resulting from heat-induced biophysical changes in cardiac tissue such as interstitial edema, hyperemia, conformational changes, cellular shrinkage and tissue coagulation. Reviewing this general inventory of effects in the context of parameters detectable by MR1, acute interstitial edema is most likely responsible for the hyperintense regions representing the area of damage observed by T2-weighted FSE imaging. The edema response is mediated by the release of vasoactive polypeptides from local inflammatory cells within seconds of the injury, which causes water and proteins to escape through gaps in the endothelial cells lining the vessel and enter the interstitial space. This near instantaneous local increase in the number of unbound protons increases the T2-relaxation constant of the tissue and gives rise to the hyperintense regions that appear to represent the spatial extent of the anatomic lesion. Additionally, lesion detection 1-2 minutes following ablation with subsequent formation over 10-15 minutes is consistent with the temporal physiologic response of local acute interstitial edema.

Contrast-Enhanced Fast Gradient Recall Echo Imaging. Although ablation lesions were not visible by T1-FGRE imaging alone, the spatial extent of the lesion was very clearly demarcated with this sequence following peripheral administration of gadolinium-DTPA. This enhancement is distinctly different from the dynamic lesion detection described for T2-FSE images and can be explained by considering the physical and physiologic mechanisms by which gadoliniurn achieves enhanced signal intensity in injured myocardium. Gadolinium-DTPA exerts its signal-enhancing effect by interacting with water protons and inducing a shorter T1 relaxation time. In uninjured myocardium, this large molecule cannot penetrate cellular membranes and is therefore restricted to the extracellular space. After endocardial ablation, however, damaged/ruptured cellular membranes allow penetration of the contrast agent into the intracellular space, significantly increasing the volume of distribution for the contrast agent and resulting in a “brighter” voxel of tissue on T I-weighted images.

For practical implementation, FGRE imaging is preferable to FSE for cardiac ablation therapy since imaging times are decreased significantly and quality images may be acquired without cardiac gating and breath-holds. An important parameter for contrast-enhanced lesion imaging is the duration post-ablation for optimal gadoliniurn uptake. In this study we injected contrast 30 minutes post-ablation and observed a rapid uptake of gadolinium in the affected area of the myocardium. It is not known, however, how quickly the lesion is capable of contrast uptake. The answer to this question has direct clinical implications and may also lend additional insight into the biophysical mechanisms of in vivo lesion formation.

Comparison With Other Imaging Modalities

Several studies have demonstrated the utility of intracardiac ultrasound for guiding cardiac ablation therapy and visualizing thermal lesions in vitro. A recent study by Epstein and colleagues compared intracardiac ultrasound to fluoroscopy guidance for creating linear right atrial lesions in a canine model and showed that intracardiac; ultrasound significantly improved targeting, energy delivery and lesion formation. While these reports are promising, the limitations of this approach include relatively poor spatial resolution, only limited views of the left and right atrium, the inability to distinguish multiple intracardiac catheters, the need for complementary x-ray fluoroscopy and the inability to accurately quantify the spatial extent of the thermal damage in vivo. Direct in vivo visualization of right atrial anatomy and radiofrequency lesions using fiberoptic probes has also been performed successfully where thermal damage is monitored based upon heat-induced myocardial color changes. In addition to the relatively small field of view produced by the probe, this methodology is subjective and does not accurately represent irreversibly damaged tissue.

While MRI guided ablation is not subject to the aforementioned limitations, the technique and system are in the early stages of development and there are number of technical requirements including non-magnetic catheters, monitoring equipment and electromagnetic filtering systems. Additionally, while new advances in scanner hardware have allowed for realtime MR imaging (20 frames/second), passive catheter tracking can be confounded by complex catheter movements that cause the catheter to leave the imaging plane. Lastly, the delayed nature of lesion formation following the initial RIF delivery confounds instantaneous assessment of lesion size.

Clinical Implications

While the approach described in this report has application for all cardiac arrhythmias curable by radiofrequency ablation, it may be particularly well-suited for more complex arrhythmias that require the accurate placement of multiple, linearly arranged lesions rather than ablation of a single focus (e.g., atrial flutter, ventricular tachycardia complicating coronary artery disease and reentrant atrial tachycardia following surgery for congenital cardiac disease). The area of highest potential impact for MR guided interventional electrophysiology, however, is in the management of atrial fibrillation. In addition to improved anatomic targeting of critical focal sites, the ability to directly visualize the spatial extent of atnial lesions with high spatial resolution may help facilitate the placement of linear transmural atrial lesions and allow for realtime interactive detection and elimination of skip lesions. This potential may have particular importance since it has been shown that ablation lines with skip lesions are not only ineffective but may be arrhythmogenic. In addition, the ability to characterize the temporal evolution of lesions can be used for therapy titration and avoidance of damage to tissue outside the ablation target volume, although the observed delayed biophysical response of the lesion may confound an instantaneous assessment of lesion size. These combined advantages may reduce the number of lesions required for conduction block, reduce procedure times and reduce the risk of perforation, all without ionizing radiation.

### CONCLUSIONS

These studies have demonstrated that radiofrequency cardiac ablation can be performed under MR1 guidance in vivo. Catheters are clearly defined and easily positioned in gradient echo images and the spatial and temporal extent of ventricular ablation lesions can be accurately visualized using T2-weighted fast spin echo imaging and T1-weighted contrast-enhanced fast gradient echo imaging with a standard cardiac phased array thoracic coil. Additionally, lesion size by MRI agrees well with actual postmortem lesion size and high fidelity intracardiac electrophysiologic signals can be acquired and monitored during imaging. MRI guided cardiac ablation may be a useful technique that will eliminate ionizing radiation exposure, help provide accurate therapy titration and facilitate the creation of linear, contiguous and transmural lesions, and may lend insight into the physiologic effects of novel ablation techniques and technologies.

What is claimed is:

1. An ablation catheter which is compatible with MRI systems, comprising a shaft and a distal tip assembly, each being made of MRI compatible material, at least one electrode supported on said tip assembly, said electrode being made of MRI compatible material, and at least one wire connected to said electrode and extending from said electrode to the proximal end of said shaft, said wire being made of MRI compatible material.

2. An ablation catheter according to claim 1, wherein said catheter is steerable and includes a plurality of steering cables extending through said shaft and distal tip assembly, said steering cables being made of MRI compatible material.

3. An ablation catheter according to claim 1, wherein said shaft is made of one of polyurethane tubing, braided extrusion and woven Dacron.

4. An ablation catheter according to claim 1, wherein said shaft is made of polyurethane tubing and braided extrusion.

5. An ablation catheter according to claim 1, wherein said shaft is made of polyurethane tubing and woven Dacron.

6. An ablation catheter according to claim 1, wherein said shaft is made of polyurethane tubing, braided extrusion and woven Dacron.

7. An ablation catheter according to claim 1, wherein said wire is made of one of copper, copper alloy and copper beryllium.

8. An ablation catheter according to claim 1, further including a snap-fit distal assembly made of MRI compatible material.

9. An ablation catheter according to claim 8, wherein said snap-fit distal assembly is made of polyurethane.

10. An ablation catheter according to claim 8, wherein said snap-fit distal assembly includes a plurality of engagable components providing a snap-fit structure, said engagable components being made of intrinsically compressible plastic.

11. An ablation catheter according to claim 10, wherein said engageable components are made of one of polycarbonate and ULTEM®.

12. An ablation catheter according to claim 7, wherein said snap-fit distal assembly further includes a core into which a potting compound is injected.

13. An ablation catheter according to claim 12, wherein said core is contained within a distal tip electrode.

14. An ablation catheter according to claim 12, wherein said potting compound is TRA-BOND FDA-2 epoxy.

15. An ablation catheter according to claim 1, further including a temperature sensor and a temperature sensor conductive wire made of MRI compatible material, and said temperature sensor conductive wire extends from said temperature sensor to the proximal end of said shaft.

16. An ablation catheter according to claim 15, wherein said temperature sensor conductive wire is made of copper and Constantine.

17. An ablation catheter according to claim 1, wherein said shaft has a proximal portion and said catheter further includes a stiffening spring in said proximal portion of said shaft, said stiffening spring being made of MRI compatible material.

18. An ablation catheter according to claim 17, wherein said stiffening spring is made of brass.

19. An ablation catheter which is compatible with MRI systems, comprising:
a shaft and a distal tip assembly, each being made of MRI compatible material, said shaft having a proximal portion;
an electrode supported on said tip assembly, said electrode being made of MRI compatible material, and a first wire connected to said electrode and extending from said electrode to the proximal end of said shaft, said first wire being made of MRI compatible material;
said catheter being steerable and including a plurality of steering cables extending through said shaft and said distal tip assembly, said steering cables being made of MRI compatible material;
a snap-fit distal assembly made of MRI compatible material;
a stiffening spring in said proximal portion of shaft, said stiffening spring being made of MRI compatible material; and
a temperature sensor being made of MRI compatible material and a second wire connected to said temperature sensor and extended from said temperature sensor to said proximal portion of said shaft, said second wire being made of MRI compatible material.

20. An ablation catheter according to claim 19 wherein
said shaft is made of one of polyurethane tubing, woven Dacron and braided extrusion;
said first wire is made of one of copper, copper alloy and copper beryllium;
at least one of said steering cables is made of stainless steel;
at least one component of said snap-fit distal assembly is made of polyurethane;
said stiffening spring is made of brass; and
said second wire is made of copper and Constantine.

21. An ablation catheter which is compatible with MRI systems, comprising a shaft and a distal tip assembly, each being made of a nonmagnetic material, at least one electrode supported on said tip assembly, said electrode being made of a nonmagnetic material, and at least one wire connected to said electrode and extending from said electrode to the proximal end of said shaft, said wire being made of a nonmagnetic material.

22. An ablation catheter according to claim 21, wherein said catheter is steerable and includes a plurality of steering cables extending through said shaft and distal tip assembly, said steering cables being made of a nonmagnetic material.

23. An ablation catheter according to claim 21, wherein said shaft is made of one of polyurethane tubing, braided extrusion and woven Dacron.

24. An ablation catheter according to claim 21, wherein said shaft is made of polyurethane tubing and braided extrusion.

25. An ablation catheter according to claim 21, wherein said shaft is made of polyurethane tubing and woven Dacron.

26. An ablation catheter according to claim 21, wherein said shaft is made of polyurethane tubing, braided extrusion and woven Dacron.

27. An ablation catheter according to claim 21, wherein said wire is made of one of copper, copper alloy and copper beryllium.

28. An ablation catheter according to claim 21, further including a snap-fit distal assembly made of a nonmagnetic material.

29. An ablation catheter according to claim 28, wherein said snap-fit distal assembly is made of polyurethane.

30. An ablation catheter according to claim 28, wherein said snap-fit distal assembly includes a plurality of engagable components providing a snap-fit structure, said engagable components being made of intrinsically compressible plastic.

31. An ablation catheter according to claim 30, wherein said engageable components are made of one of polycarbonate and ULTEM®.

32. An ablation catheter according to claim 27, wherein said snap-fit distal assembly further includes a core into which a potting compound is injected.

33. An ablation catheter according to claim 32, wherein said core is contained within a distal tip electrode.

34. An ablation catheter according to claim 32, wherein said potting compound is TRA-BOND FDA-2 epoxy.

35. An ablation catheter according to claim 21, further including a temperature sensor and a temperature sensor conductive wire made of a nonmagnetic material, and said temperature sensor conductive wire extends from said temperature sensor to the proximal end of said shaft.

36. An ablation catheter according to claim 35, wherein said temperature sensor conductive wire is made of copper and Constantine.

37. An ablation catheter according to claim 21, wherein said shaft has a proximal portion and said catheter further includes a stiffening spring in said proximal portion of said shaft, said stiffening spring being made of a nonmagnetic material.

38. An ablation catheter according to claim 37, wherein said stiffening spring is made of brass.

39. An ablation catheter which is compatible with MRI systems, comprising:
a shaft and a distal tip assembly, each being made of a nonmagnetic material, said shaft having a proximal portion;
an electrode supported on said tip assembly, said electrode being made of a nonmagnetic material, and a first wire connected to said electrode and extending from said electrode to the proximal end of said shaft, said first wire being made of a nonmagnetic material;
said catheter being steerable and including a plurality of steering cables extending through said shaft and said distal tip assembly, said steering cables being made of a nonmagnetic material;
a snap-fit distal assembly made of a nonmagnetic material;
a stiffening spring in said proximal portion of shaft, said stiffening spring being made of a nonmagnetic material; and
a temperature sensor being made of a nonmagnetic material and a second wire connected to said temperature sensor and extended from said temperature sensor to said proximal portion of said shaft, said second wire being made of a nonmagnetic material.

40. An ablation catheter according to claim 39 wherein
said shaft is made of one of polyurethane tubing, woven Dacron and braided extrusion;
said first wire is made of one of copper, copper alloy and copper beryllium;
at least one of said steering cables is made of stainless steel;
at least one component of said snap-fit distal assembly is made of polyurethane;
said stiffening spring is made of brass; and
said second wire is made of copper and Constantine.
